(19) United States
(12) Patent Application Publication
(10) Pub. No.: US 2025/0332319 A1
(43) Pub. Date: Oct. 30, 2025

(54) POLYMER SYSTEM FOR FORMING A HYDROGEL

(71) Applicant: STELLENBOSCH UNIVERSITY, Western Cape Province (ZA)

(72) Inventors: Lubertus KLUMPERMAN, Western Cape Province (ZA); Leanne KELLERMANN, Western Cape Province (ZA)

(21) Appl. No.: 18/867,333

(22) PCT Filed: May 19, 2023
(86) PCT No.: PCT/ZA2023/050027

§ 371 Date: Nov. 19, 2024

(30) Foreign Application Priority Data
May 19, 2022 (GB) 2207359.7

(51) Int. Cl. A61L 24/00; A61L 24/04; A61L 24/06

(52) U.S. Cl. CPC A61L 24/0031; A61L 24/046; A61L 24/06

(57) ABSTRACT

A polymer system for forming a hydrogel and a male contraceptive method of reversibly occluding a vas deferens with the hydrogel are provided. The polymer system comprises a water-soluble and at least partially thiolated poly(styrene-co-maleic acid) polymer and a water-soluble cross-linking agent with suitable reactive functional groups at its ends which are configured to crosslink to the thiol groups of the thiolated poly(styrene-co-maleic acid) polymer, when aqueous solutions of the at least partially thiolated poly(styrene-co-maleic acid) polymer and cross-linking agent are combined to form the hydrogel in use. The hydrogel includes dynamic covalent bonds which can be broken with an appropriate solution to dissolve the hydrogel in situ.

### CROSS-REFERENCE(S) TO RELATED APPLICATIONS

This application claims priority from United Kingdom patent application number GB2207359.7 filed on 19 May 2022, which is incorporated by reference herein.

### FIELD OF THE INVENTION

This invention relates to a polymer system for forming a hydrogel and methods of preparing the polymer system. In particular, it relates to a biocompatible hydrogel which can be injected into the vas deferens to serve as a reversible male contraceptive.

### BACKGROUND TO THE INVENTION

Biologically compatible gels have wide-ranging biomedical applications including tissue engineering, drug or gene delivery, 3D bioprinting, wound healing, antimicrobial applications, and contraception.

The main contraceptive methods currently available to men are condoms and vasectomy, with the latter being more reliable but irreversible. Polymer gels such as reversible inhibition of sperm under guidance (RISUG) of U.S. Pat. No. 5,488,075, and Vasalgel™ have been developed as alternative male contraceptive methods. These polymer gels are injected directly into the male vas deferens and serve as contraceptive devices by mainly obstructing the transport of sperm from the testes via the vas deferens.

RISUG involves the use of a copolymer of styrene and maleic anhydride (SMAh) (Mw≈60,000-100,000 g/mol), dissolved in dimethyl sulfoxide (DMSO), and injected directly into the vas deferens following two small incisions. The proposed mode of action is a combination of vas occlusion, pH lowering and charge disturbance which affect the fertilizing ability of sperm. Once injected into the vas deferens, it has been suggested that SMAh converts to poly(styrene-co-maleic acid) (SMA), due to exposure to spermatic fluid, and the DMSO is absorbed into the surrounding tissue. This leaves a hydrogel within the folds of the vasa, composed of a protein-SMA agglomerate. When RISUG is used as a vas occlusive device a significant amount of pressure is required to infuse the viscous RISUG material into the narrow lumen of the vas deferens, resulting in post-operative complications. Animal and human trials showed efficacy of RISUG as a contraceptive. Intravasal injections of a sodium bicarbonate (NaHCO₃) solution were used to try and reverse occlusion. However, compromised epididymal and vas deferens function strongly indicates that this procedure is not safely reversible.

Vasalgel™ is based on higher molecular weight poly(styrene-co-maleic acid) polymers (Mw≈330 000 g/mol), which are also dissolved in DMSO and injected directly into the narrow lumen of the vas deferens. Intravasal injections of a NaHCO₃ solution have also been used in attempts to reverse occlusion of Vasalgel™ in rabbits. However, as with RISUG, the Vasalgel™ high molecular weight polymer gel acts like a viscous polymer plug that is difficult to infuse into the lumen and to redissolve, thus having poor reversibility.

Accordingly, there is a need for polymer compositions or systems that can be used in a reliable, safe and reversible contraceptive method, amongst other potential biological or medical applications.

The preceding discussion of the background to the invention is intended only to facilitate an understanding of the present invention. It should be appreciated that the discussion is not an acknowledgment or admission that any of the material referred to was part of the common general knowledge in the art as at the priority date of this application.

### SUMMARY OF THE INVENTION

In accordance with an aspect of the invention there is provided a polymer system for forming a hydrogel, the system comprising a water-soluble and at least partially thiolated poly(styrene-co-maleic acid) polymer; and a water-soluble cross-linking agent with reactive functional groups at its ends which are configured to crosslink to the thiol groups of the thiolated poly(styrene-co-maleic acid) polymer when aqueous solutions of the at least partially thiolated poly(styrene-co-maleic acid) polymer and the cross-linking agent are combined to form the hydrogel in use.

The reactive functional groups of the crosslinking agent may be activated esters. The activated esters may be succinimidyl valerate or pentafluorophenyl ester groups. The cross-linking agent may be a telechelic polymer. The telechelic polymer may be any water-soluble, biocompatible, chain end functionalised polymer such as polyether (e.g., poly(ethylene glycol) or poly(ethylene glycol-co-propylene glycol) and the like) or vinyl polymer (e.g. poly(N-vinylpyrrolidone), poly(2-hydroxyethyl methacrylate and the like). The telechelic polymer may be poly(ethylene glycol bis-succinimidyl valerate). The poly(ethylene glycol bis-succinimidyl valerate) may have a number average molecular weight (Mₙ) of between 1,500 and 3,500 g/mol.

The at least partially thiolated poly(styrene-co-maleic acid) polymer may have a number average molecular weight (Mₙ) that is between 10,000 and 85,000 g/mol. The at least partially thiolated poly(styrene-co-maleic acid) polymer may have a narrow molecular weight distribution characterised by a dispersity (Ð) of less than 2, preferably less than or equal to 1.5 as determined by size exclusion chromatography (SEC).

The at least partially thiolated poly(styrene-co-maleic acid) polymer may be at least partially thiolated poly(styrene-alt-maleic acid) polymer, which may be prepared from an alternating poly(styrene-alt-maleic anhydride) base polymer via a controlled radical polymerisation process such as reversible addition-fragmentation chain transfer (RAFT) polymerisation. The thiolating agents can be cysteine, cysteamine, but also other compounds that contain a primary amine and a thiol. The thiol grafting ratio of cysteamine to maleic acid of the thiolated poly(styrene-alt-maleic acid) polymer may be between about 0.1 and 0.8, and preferably between about 0.4 and 0.8.

The at least partially thiolated poly(styrene-co-maleic acid) polymer and the cross-linking agent may each be independently present in separate aqueous solutions. The separate aqueous solutions may be different buffer solutions, optionally having different pH values. The solution of the at least partially thiolated poly(styrene-co-maleic acid) polymer may have a pH between 8 and 9, whereas the solution of the cross-linking agent may have a pH between 7 and 8. When the cross-linking agent is a telechelic polymer, each buffer solution may include between 10% and 40% (w/w) of polymer, preferably between 25% and 35% (w/w) of polymer.

In accordance with a second aspect of the invention, there is provided a hydrogel formed from the above-described polymer system. The hydrogel may be formed with a polymer system having a 1:1 molar ratio of thiol (sometimes also referred to as sulfhydryl) groups to reactive functional groups. The hydrogel may have a swelling ratio of between 10 and 40 g/g ex situ, preferably between 20 and 30 g/g, as determined by equilibrium swelling studies in phosphate buffered saline (PBS).

In accordance with a third aspect of the invention, there is provided a method of preparing the above-described polymer system, the method comprising the steps of:
modifying a poly(styrene-co-maleic anhydride) polymer with a thiolating agent as described before like cysteamine to form an at least partially thiolated poly(styrene-co-maleic anhydride) polymer;
hydrolysing the at least partially thiolated poly(styrene-co-maleic anhydride) polymer to form the thiolated poly(styrene-co-maleic acid) polymer; and
separately providing or preparing the cross-linking agent, thereby forming the polymer system.

Cysteamine hydrochloride may be used to modify the poly(styrene-co-maleic anhydride) polymer. The molar ratio of the thiolating agent, like cysteamine, to maleic anhydride used during this modification may range between 0.1:1 and 0.8:1.

The poly(styrene-co-maleic anhydride) polymer may be synthesised by controlled radical polymerisation to obtain a poly(styrene-co-maleic anhydride) polymer with a narrow molecular weight distribution characterised by a dispersity (Ð) of less than 2, preferably less than or equal to 1.5. The method accordingly may actively include the step of synthesising the a poly(styrene-co-maleic anhydride) polymer with a narrow molecular weight distribution characterised by a dispersity (Ð) of less than 2, preferably less than or equal to 1.5, by controlled radical polymerisation.

The cross-linking agent may be a water-soluble polymer with the indicated functional end-groups, preferably poly(ethylene glycol bis-succinimidyl valerate) prepared by end group functionalisation of poly(ethylene glycol).

In accordance with a fourth aspect of the invention, there is provided a method of vas-occlusive contraception, the method comprising providing a hydrogel formed from the above-described polymer system or a hydrogel prepared according to the above-described method in a lumen of a vas deferens in a living subject.

The insertion or formation of the hydrogel into or in the lumen leads to sterility in the subject. Aqueous solutions of the at least partially thiolated poly(styrene-co-maleic anhydride) polymer and the cross-linking agent may be separately administered into the lumen. Alternatively, aqueous solutions of the at least partially thiolated poly(styrene-co-maleic anhydride) polymer and the cross-linking agent may be administered concurrently into the lumen. As soon as both the at least partially thiolated poly(styrene-co-maleic anhydride) polymer and the cross-linking agent are present in the lumen they reversibly crosslink into a contraceptive hydrogel.

At the time or moment that fertility is desired to be regained, a solution, preferably an aqueous solution, configured to dissolve the hydrogel present in the lumen to reverse the occlusion of the lumen may optionally be administered into the lumen. The solution configured to dissolve the hydrogel may be an aqueous thiolate solution such as a cysteamine methyl ester (CME) solution. An aqueous CME solution may be used to dissolve the hydrogel in the lumen when the reactive functional groups on the cross-linking agent are activated esters which in use form thioester linkages in the reversibly crosslinked hydrogel. The CME solution may have a concentration ranging between 0.1 M and 4 M, preferably between 0.5 M and 3 M, or more preferably a concentration of 2.5 M. The CME solution may be a CME-HCl solution with a pH adjusted to between 8 and 9, preferably with a pH of 8.5.

In accordance with a fifth aspect of the invention, there is provided a kit for forming a hydrogel comprising the above-described polymer system.

The at least partially thiolated poly(styrene-co-maleic acid) polymer and the cross-linking agent of the system provided in the kit may each be independently present in separate aqueous solutions. The kit may further include a dual-barrel cartridge or syringe configured to concurrently or simultaneously administer the separate aqueous solutions of the at least partially thiolated poly(styrene-co-maleic acid) polymer and the cross-linking agent. The dual-barrel cartridge or syringe may be preloaded with a first cartridge or barrel of the dual-barrel cartridge or syringe containing the aqueous solution of the at least partially thiolated poly(styrene-co-maleic acid) polymer and a second cartridge or barrel of the dual-barrel cartridge or syringe containing the aqueous solution of the cross-linking agent.

Embodiments of the invention will now be described, by way of example only, with reference to the accompanying drawings.

### BRIEF DESCRIPTION OF THE DRAWINGS

In the drawings:

FIG. 1 is a schematic illustration of a kit for forming a hydrogel with two separate aqueous solutions in a dual-barrel syringe;

FIG. 2 is a plot of the gelation time against the degree of cysteamine grafting on thiolated poly(styrene-co-maleic acid) (SMA-SH) polymers gelled with poly(ethylene glycol) bissuccinimidyl valerate polymers of different molecular weights (PEG-bis-SV1,500 and PEG-bis-SV3,350), respectively, reacted at room temperature;

FIG. 3 is a bar graph showing the swelling ratio, S (g/g), of hydrogel formulations soaked in a phosphate buffered saline (PBS) buffer solution (pH=7.4);

FIG. 4 is a plot of the swelling ratio (g/g) against the degree of cysteamine grafting on SMA-SH polymers;

FIG. 5 is a graph (a) of the results of amplitude sweep tests versus sheer strain (γ) for hydrogel formulation H1 of Table 2;

FIG. 6 is a graph (b) of the results of amplitude sweep tests versus sheer strain (γ) for hydrogel formulation H2 Table 2;

FIG. 7 is a graph (c) of the results of amplitude sweep tests versus sheer strain (γ) for hydrogel formulation H3 of Table 2;

FIG. 8 is a graph (d) of the results of amplitude sweep tests versus sheer strain (γ) for hydrogel formulation H4 of Table 2

FIG. 9 is a graph of the results of amplitude sweep tests of hydrogel formulations, H5 to H14 of Table 2, to determine the linear viscoelastic region (LVER) from G′ (storage modulus) and G″ (loss modulus);

FIG. 10 is a plot of the linear viscoelastic region (LVER), as determined by the strain dependence of G′ (storage modulus) and G″ (loss modulus), for high molecular weight poly(styrene-alt-maleic acid) (HMW SMAcid) in DMSO;

FIG. 11 is a graph of the storage modulus (G′) and loss modulus (G″) versus angular frequency (ω) (strain determined by LVER) for hydrogels formulations, H5 to H14;

FIG. 12 is a graph of the storage modulus (G′) and loss modulus (G″) versus angular frequency (ω) (strain determined by LVER) for hydrogels formulations H8 to H10 formed with SMA 3;

FIG. 13 is a graph showing the loss factor (tan δ) versus angular frequency (ω) for H1 to H5;

FIG. 14 is a graph showing the loss factor (tan δ) versus angular frequency (ω) for H6 to H14; and

FIG. 15 is several microscope images of haematoxylin and eosin (H&E) stained sections of (a) unchanged male vas, (b) vas injected with high molecular weight poly(styrene-alt-maleic acid) (HMW SMAcid), dissolved in DMSO, and (c) reversal of HMW SMAcid by flushing of the vas with sodium bicarbonate solution; and

is several microscope images of vas deferens sections, stained with H&E, when (a) injected with hydrogel formulation H9, (b) H9 reversal with CME solution, (c) injected with hydrogel formulation H13, and (d) H13 reversal with CME solution.

### DETAILED DESCRIPTION WITH REFERENCE TO THE DRAWINGS

Hydrogels are highly hydrated, three-dimensional crosslinked polymeric networks. A system for forming an injectable, biocompatible and dynamic hydrogel with appropriate swelling and viscoelastic properties for use in biomedical applications such as reversible contraception in subjects is provided. The subjects may be male or female, preferably male, and may be animals or humans. The polymer system comprises at least two components, a biocompatible thiol-grafted styrene maleic acid (SMA) based polymer and an appropriately functionalised cross-linking agent which together can form a dynamically and reversibly crosslinked hydrogel matrix. The cross-linking agent may be a water-soluble, telechelic polymer. The two components of the system may be provided separately, each component being independently soluble in aqueous solution such that the hydrogel may ideally be formed in situ under physiological conditions within the body of a subject, particularly within the lumen of the vas deferens when used in male subjects. When the hydrogel is formed in situ it substantially conforms to the shape of the body part, i.e, the folded shape of the lumen. The pH of the aqueous solutions may be controlled to optimise gelling efficiency thereby avoiding absorption or degradation of any low molecular weight polymer components in the body.

The dynamic hydrogel formed through reversible covalent bonds is capable of “on-demand” dissolution. The reversibly crosslinked low molar mass SMA-based polymer is readily dissolvable upon administration of a dissolution agent capable of “uncrosslinking” the hydrogel, so that the system returns to a low molar mass polymer with a concomitant low viscosity. In this manner the hydrogel may find use as a reversible male contraceptive method when it is formed in the lumen of the vas deferens when contraception is required and dissolved later to re-establish fertility. The viscoelastic properties of the hydrogel compositions formed with the polymer system may be tailored so that damage to the lumen membrane and any surrounding tissues is avoided. Such tailoring of the viscoelastic properties can be carried out through variation of the molecular weight of the poly(styrene-co-maleic anhydride) starting material; and/or through variation of the degree of modification of the partially thiolated poly(styrene-co-maleic anhydride) polymer; and/or through variation of the chain length of the water-soluble cross-linking agent.

Accordingly, a polymer system for forming a biocompatible hydrogel is provided which comprises a water-soluble, at least partially thiolated poly(styrene-co-maleic acid) (SMA-SH) polymer; and a water-soluble cross-linking agent, preferably a water-soluble, telechelic polymer. The cross-linking polymer has reactive functional groups at its chain ends which are configured to crosslink to the thiol groups of SMA-SH polymer when aqueous solutions of the SMA-SH polymer and cross-linking agent are combined and/or mixed to form the hydrogel in use. The reactive functional groups may be selected from the group of activated esters (to form thioester linkages), thiol groups (to form disulphide bridges) or acrylates (for Michael addition). Activated esters are preferred and may, for example, be succinimidyl valerate or pentafluorophenyl ester groups amongst others. The activated ester groups can crosslink to the thiol groups on the SMA-SH polymer to form a dynamically and reversibly crosslinked thioester hydrogel with dynamic covalent bonds which can readily be broken or replaced in the presence of a suitable agent. In the case of thioester crosslinks being present in the hydrogel, a thiolate solution may be used to dissolve the hydrogel, preferably a cysteamine methyl ester (CME) solution.

The crosslinking agent may be a telechelic polymer composed of any water-soluble, biocompatible, chain end functionalised polymer such as polyether (e.g. poly(ethylene glycol) or poly(ethylene glycol-co-propylene glycol) or the like) or vinyl polymer (e.g. poly(N-vinylpyrrolidone), poly(2-hydroxyethyl methacrylate or the like).

In one embodiment, the cross-linking agent is poly(ethylene glycol bis-succinimidyl valerate) (PEG-bis-SV). Poly(ethylene glycol) (PEG) is a biocompatible polymer with hydroxyl end-groups susceptible to chain-end derivatisation. The PEG-bis-SV may be prepared from PEG having a number average molecular weight of between about 1,500 and 3,500 g/mol, preferably between about 3,000 and 3,500 g/mol, and most preferably about 3,350 g/mol, via a stepwise derivatisation process.

The at least partially thiolated SMA-SH polymer has a relatively low molecular weight which makes it more readily dissolvable upon exposure to a suitable dissolution agent capable of breaking the dynamic covalent crosslinking bonds. The at least partially thiolated SMA-SH polymer may be prepared from a precursor poly(styrene-co-maleic anhydride) (SMAh) polymer having a number average molecular weight that is between about 10,000 and about 85,000 g/mol. The precursor SMAh polymer is preferably synthesized via controlled radical polymerisation technique such as RAFT mediated polymerisation to better control chain length and polymer architecture. A well-defined polymeric architecture is important for SMAh's biocompatibility, quantitative functionalisation, and induced hydrophilicity. Dispersity (Ð) is defined as the ratio of the weight-average molar mass (M_{w}) to the number-average molar mass (Mₙ) and is a measure of the heterogeneity of sizes of molecules in a mixture. The dispersity is determined by size exclusion chromotagraphy using N,N-dimethylformamide (DMF) as mobile phase as described in Scheuing, D. R., Size exclusion chromatography of polyelectrolytes in dimethylformamide. J. Appl. Polym. Sci., 1984, 29, 819-2828, which is incorporated herein by reference thereto. Commercially available SMAh products, synthesised via conventional radical polymerisation, have a dispersity of about 2. When controlled radical polymerisation is used to prepare the precursor polymer, it and the at least partially thiolated SMA polymer prepared therefrom have a narrower molecular weight distribution characterised by a dispersity (Ð) of less than 2, preferably less than or equal to about 1.5, more preferably less than or equal to about 1.3, as determined by size exclusion chromatography (SEC) with DFM as eluent and calibrated using poly(methyl methacrylate) (PMMA) standards.

It is also preferred that the precursor SMAh polymer is a substantially alternating copolymer in which styrene centred triads are flanked by two MAh units (MSM structure). RAFT polymerisation with a feed ratio of monomers at 1:1 (Sty:MAh) typically produces such a strong alternating system. Accordingly, the at least partially thiolated poly(styrene-co-maleic acid) polymer prepared from RAFT-synthesised SMAh is an alternating polymer, i.e. a poly(styrene-alt-maleic acid) polymer.

The thiol groups may be introduced onto the synthesized SMAh copolymers through thiol, preferably cysteamine grafting. The thiol grafting ratio of cysteamine to maleic acid of the at least partially thiolated poly(styrene-co-maleic acid) may range between about 0.1 and 0.8, preferably between about 0.4 and 0.8. The remaining maleic anhydride units are then ring-opened to afford a water-soluble, thiol-containing polymer, SMA-SH.

The at least partially thiolated SMA-SH polymer and the cross-linking agent, which may be a telechelic polymer, can each be independently present and dissolved in separate aqueous solutions which are combined to form the hydrogel via a crosslinking reaction between thiolate anions and the reactive functional groups on the telechelic polymer, such as the activated ester succinimidyl valerate groups as shown in Scheme 1.

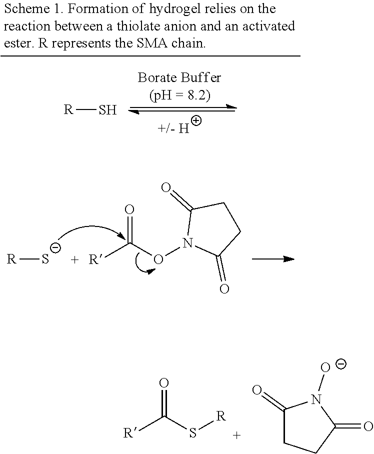

In the crosslinking reaction, a nucleophilic thiolate attacks the activated carbonyl on the crosslinker. Thiols are practically unreactive as nucleophiles and react 10¹⁰-times slower than the corresponding thiolates. Since a thiolate must form prior to crosslinking, the pH is an important factor in hydrogel precursor compositions or formulations. The separate aqueous solutions of the SMA-SH and the cross-linking agent may accordingly be buffered solutions of different pH values. When the cross-linking agent is a telechelic polymer, each buffer solution may include between about 10% and 40% (w/w) of polymer, preferably between about 25% and 35% (w/w) of polymer, more preferably about 30% (w/w) of polymer. For example, the SMA-SH polymer may be in a basic solution such as a 5 mM borate buffer solution (pH=8.2) with 5 mM dithiothreitol (DTT). A PEG-bis-SV crosslinking polymer can be made up to a 30% (w/w) polymer solution in phosphate-buffered saline (PBS) at pH 7.4. Where the different pH values are chosen to provide optimum solubility and stability of the individual polymer solutions.

A dynamically and reversibly crosslinked hydrogel may be formed from the polymer system consisting of a water-soluble, at least partially thiolated SMA-SH polymer, and a water-soluble cross-linking agent, which may be PEG-bis-SV, for example, as demonstrated in Scheme 2.

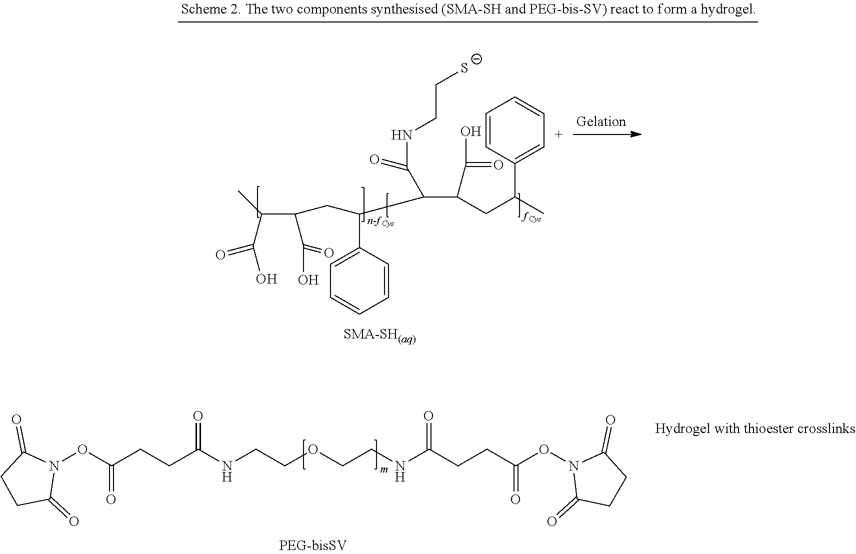

The hydrogel is formed with a polymer system having a 1:1 molar ratio of thiol groups to reactive functional groups. The molar ratio of thiol to N-succinimidyl succinate in Scheme 2 is accordingly kept at 1:1. Generally, this molar ratio is selected between 0.9-1 and 1.1-1.

The thioester bond is a suitable form of crosslinking for use in a reversible contraceptive method due to its rapid in situ formation, stability, and on-demand dissolution upon exposure to an extraneous, biocompatible thiolate solution, such as an CME solution.

To act as a contraceptive, the hydrogel should hinder and/or fully obstruct the transfer of sperm. The gel should ideally have a suitable pore size that is smaller than the head width of a sperm cell, which is about 2 μm. It was found that hydrogels formed with SMA-SH polymers having a thiol grafting molar ratios above 0.4 were impermeable to 2 μm monodisperse silica particles used to mimic sperm cell head width, with no particles trapped in the hydrogel matrix following a permeability and penetration assessment.

The hydrogels formed with SMA-SH and PEG-bis-SV may have gelation times of less than 5 minutes, and preferably less than 1 minute. The time taken to form the hydrogel decreases with an increasing degree of the thiolating x-link agent (e.g., cysteamine) grafting on the SMA-SH polymers. It was also found that gels fabricated with PEG-bis-SV having a number average molecular weight between 3,000 and 3,500 g/mol, such as 3,350 g/mol, gelled faster due to a larger radius of gyration and longer end-to-end distance scale than shorter length PEG such as those with a number average molecular weight of 1,500 g/mol.

For a hydrogel to act as an occlusive device in the body, it should be able to hold fluid and maintain a moist environment in the body. This should also translate into a good fit within the body, particularly within the highly folded lumen environment of the vas deferens. The degree of swelling depends on the crosslinking density, which is, in turn related to the pore size of the materials. The contraceptive hydrogel formed with the polymer system described herein may have having a swelling ratio of between about 10 and 40 g/g, preferably between about 20 and 30 g/g, more preferably between about 24 and 28 g/g, as determined by equilibrium swelling studies in phosphate buffered saline (PBS).

Dissolution of thioester crosslinked hydrogels is readily brought about through exposure to a solution of the biocompatible L-cysteine methyl ester (CME), or an equivalent reagent such as a 2-mercaptoethanesulfonate (MES) solution or cysteine N-terminal peptides for example, that can affect a process known as thiol-thioester exchange, also known as native chemical ligation (NCL).

The hydrogel dissolves in less than 25 minutes. Dissolution times, which are equal to or less than 25 minutes were measured in exemplary hydrogel formulations, and are related to the permeability of the formed hydrogel and the ability of the CME solution to penetrate the hydrogel's three-dimensional matrix. There is a marked increase in dissolution time with increasing degree of cysteamine functionalisation, i.e., decrease in pore size. Since hydrogel dissolution is brought about by thiol-thioester exchange as demonstrated in Scheme 3, the rate of dissolution is affected by the pH and concentration of the CME solution as well, which may be optimised.

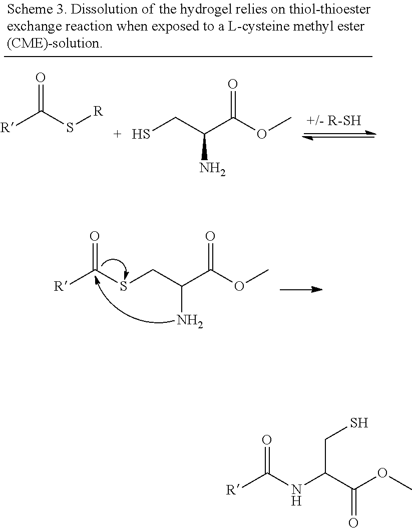

A method of preparing the above-described polymer system is further provided. The method comprises the steps of modifying a poly(styrene-co-maleic anhydride) (SMAh) polymer with cysteamine to form an at least partially thiolated poly(styrene-co-maleic anhydride) polymer; hydrolysing or ring-opening and thereby solubilising the at least partially thiolated poly(styrene-co-maleic anhydride) polymer to form the thiolated poly(styrene-co-maleic acid) polymer; and separately providing or preparing the cross-linking agent.

The maleic anhydride (MAh) in SMAh offers a reactive handle. Due to MAh's electrophilic nature, it is susceptible to nucleophilic attack, and subsequent grafting of thiol-groups onto the polymer backbone. Cysteamine hydrochloride may be used to modify the poly(styrene-co-maleic anhydride) polymer. The concentration ratio of cysteamine to maleic anhydride used during this modification may range between about 0.1:1 and 0.8:1. Hydrolysis of the thiol-grafted SMAh, lends hydrophilicity to the otherwise water-insoluble polymer. This may be achieved by refluxing the thiol-grafted SMAh in a borate buffer (pH=8.5).

A well-defined polymer architecture is necessary for biocompatibility and thiol functionalisation, and this can be achieved by synthesising a well-defined SMAh precursor polymer utilising controlled radical polymerisation such as RAFT polymerisation. In this manner, RAFT polymerisation techniques may be employed to synthesise a poly(styrene-co-maleic anhydride) polymer with a narrow molecular weight distribution characterised by a dispersity (Ð) of less than 2, preferably less than 1.5 and more preferably less than 1.3.

An exemplary RAFT polymerisation process is shown in Scheme 4.

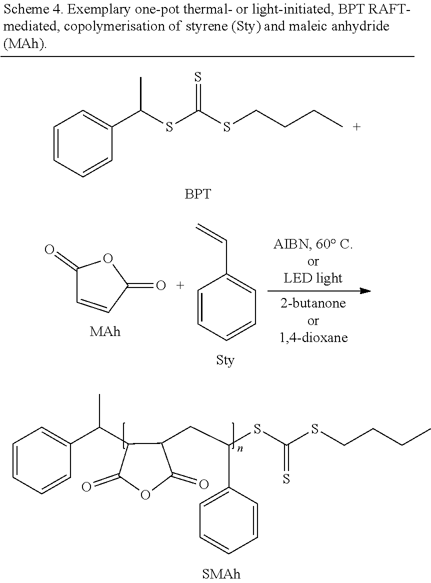

Controlled radical polymerisation of SMAh is demonstrated in Scheme 4 using butyl (1-phenylethyl) carbonotrithioate (BPT), a trithiocarbonate (TTC) compound, as CTA- or RAFT-agent. Both thermal- and visible-light-initiation methods, and both 2-butanone and 1,4-dioxane solvents, investigated provided control and gravimetric yields. However, the visible light induced RAFT polymerisation of SMAh in 1,4-dioxane, without exogeneous initiators or photocatalysts, proved to provide the best overall polymerisation rate and control.

At least some of the maleic anhydride residues of SMAh are then modified with cysteamine to produce SMA-SH. An exemplary synthetic route using cysteamine hydrochloride is shown in Scheme 5.

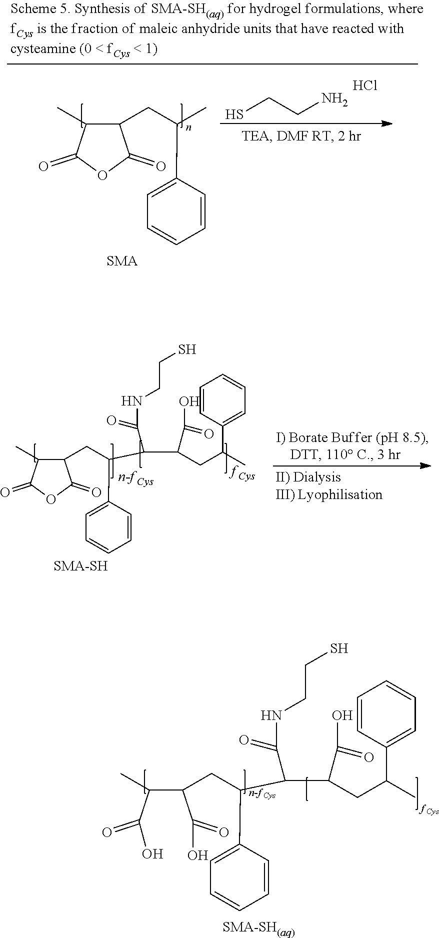

Grafting ratios (N_{Cys}) are calculated as follows:

$${N}_{Cys} = \frac{[ SH ]}{[ MAh ]}$$

where [SH] is the concentration of the sulfhydryl groups (mol/L) as may be determined by the Ellman's assay or by FTIR and [MAh] is the concentration of MAh (mol/L). It was found that Ellman's assay significantly underrepresents the thiol content in the synthesised SMA-SH polymers. This was attributed to the fact that disulphide bonds readily form in the absence of a reducing agent, i.e., the grafting ratio does not equal the free thiol present in the sample. FTIR spectroscopy was used to estimate the grafting ratio as has been done previously on the grafting of SMAh with oxazoline (M. Bruch, D. Mäder, F. Bauers, T. Loontjens and R. Mülhaupt, J. Polym. Sci. A: Polym. Chem, 2000, 38, 1222-1231).

The cross-linking agent used in the polymer system may be poly(ethylene glycol bis-succinimidyl valerate) (PEG-bis-SV) prepared by end group functionalisation of PEG. PEG is a difunctional and highly flexible polymer that is soluble in water and several organic solvents. This has the potential to lead to difficulties in the separation, isolation and purification of PEG derivatives. Reaction conditions and purification steps were optimised to obtain a PEG-bis-SV crosslinker at near quantitative conversion according to the exemplary five-step process in Scheme 6.

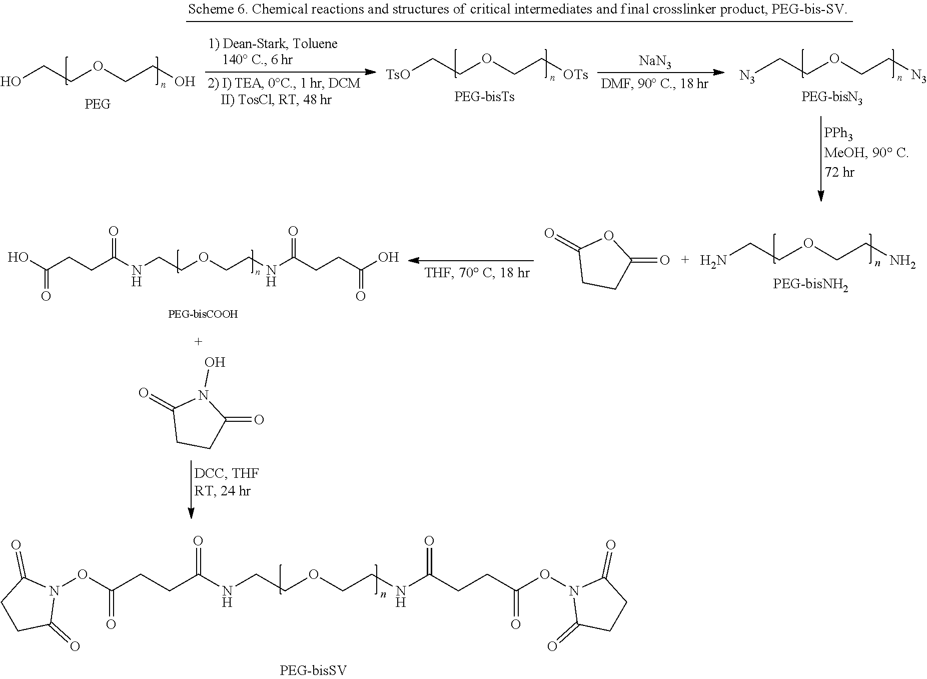

The hydrogel formed from an at least partially thiolated SMA-SH polymer and an appropriately functionalised cross-linking agent can be used as a reversible contraceptive device by reversibly occluding a part of the anatomy of the subject to prevent transfer of sperm and avoid fertilization. The contraceptive hydrogel may be provided or applied to the appropriate body part and may optionally be dissolved again by administering a solution configured to dissolve the hydrogel.

The hydrogel can, for example, be used as a male contraceptive device in a method of reversibly occluding a lumen of a vas deferens in a subject. Such a method of vas-occlusive contraception comprises providing a hydrogel formed from the polymer system described herein in a lumen of a vas deferens in a living subject or mammal, preferably in a human. The phrase “providing a hydrogel formed from the polymer system in a lumen” as used herein is meant to include both options of either administering the polymer system (the two polymer solutions that crosslink when combined) into the lumen or administering the hydrogel into the lumen. It is preferred that the polymer system is administered into the lumen so that the contraceptive hydrogel forms in situ, thereby conforming to the shape of the lumen. The vas-occlusive method of contraception is fully reversible and therefore, depending on the wishes of the subject, may include optionally administering a solution, preferably an aqueous solution, into the lumen that is configured to dissolve the hydrogel present in the lumen to reverse the occlusion of the lumen and restore fertility in use.

Administration of the polymer system or contraceptive hydrogel device into the lumen may occur under guidance of suitable imaging, such as ultrasound imaging and a needle or catheter may be inserted into the lumen to administer the polymer system into the lumen of the vas deferens through the needle or catheter. The method of vas-occlusive contraception may therefore further include one or more of the following steps (as described in more detail in WO 2017/083753 A1 which is incorporated by reference thereto herein):
(i) identifying a vas deferens of a subject in need of contraception;
(ii) placing an ultrasound probe on or near the vas deferens and administering ultrasonic energy to image a lumen portion of the vas deferens, and
(iii) performing one or more or all of the following steps:
a. measuring one or more dimensions of the lumen portion of the vas deferens;
b. percutaneously placing a needle or catheter portion thereof into the lumen portion of the vas deferens;
c. administering a selected amount of the polymer system or hydrogel described herein into the lumen portion of the vas deferens through the needle or catheter;
d. confirming formation of an occlusion inside the lumen portion, which occlusion is provided by the polymer system or hydrogel administered into the lumen; and/or
e. determining one or more dimensions of the occlusion inside the lumen portion.

In use, the hydrogel renders sperm immotile, inviable and/or infertile thereby serving as a contraceptive in male subjects.

To ensure substantial conformity of the hydrogel with the shape of the lumen, aqueous solutions of the at least partially thiolated poly(styrene-co-maleic acid) polymer and the cross-linking agent of the polymer system may be separately administered into the lumen where they reversibly crosslink or form into the contraceptive hydrogel within the lumen (i.e. in situ). The aqueous solutions of the at least partially thiolated poly(styrene-co-maleic acid) polymer and the cross-linking agent of the polymer system may be administered at different times or concurrently into the lumen. Concurrent administration is preferred so that the gelling process can start instantaneously upon contact of the two components of the polymer system when administered together into the lumen of the vas deferens.

The solution configured to dissolve the contraceptive hydrogel, generally at a later time when fertility is to be restored, may be a thiolate solution such as a CME solution when the reactive functional groups on the telechelic polymer are activated esters which form thioester linkages in the hydrogel. The lumen may be flushed with the solution to dissolve the contraceptive hydrogel.

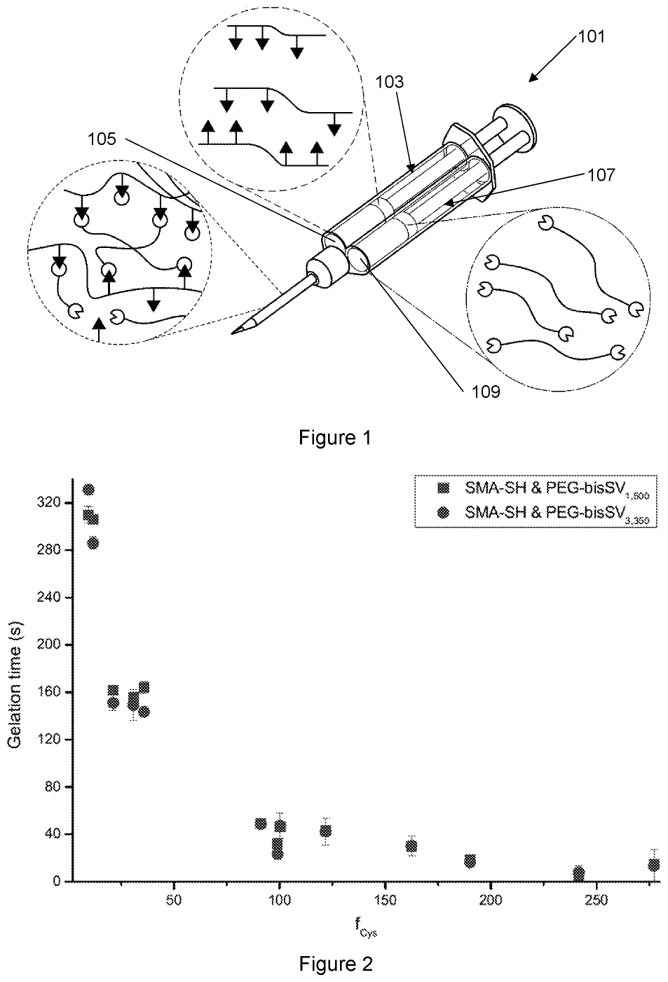

The polymer system may be provided in a kit for forming a hydrogel. The kit may be a reversible contraceptive kit to be administered or applied to or injected into a subject by a suitably qualified medical professional. The at least partially thiolated SMA-SH polymer and the cross-linking agent of the polymer system provided in the kit would preferably each be independently present and dissolved in separate aqueous solutions which may be housed in a dual-barrel cartridge or syringe (101) configured to concurrently administer the separate aqueous solutions as shown in FIG. 1. The dual-barrel cartridge or syringe may be preloaded with a first barrel (103) of the dual-barrel syringe containing the aqueous solution of the at least partially thiolated poly(styrene-co-maleic acid) polymer (105), and a second barrel (107) containing the cross-linking agent (109). Administration of both solutions with the dual-barrel cartridge or syringe (101) into a body part of a subject results in gelling or at least the completion of gelling of the hydrogel in the body part. When the hydrogel is to be used as a male contraceptive, the polymer system may be administered within the lumen of the vas deferens.

Additionally, a second kit may be provided that includes a suitable dissolution agent to dissolve the hydrogel at an arbitrary time after it has been formed. Alternatively, the dissolution agent may be provided in the first kit too.

### EXAMPLES

### Synthetic Procedures

1. General Procedure to Produce poly(styrene-co-maleic anhydride) (SMAh)

### Thermal Initiation Procedure:

A Schlenk flask containing a magnetic stirrer bar was charged with BPT (10 mg, 37 μmol, 1 eq) and AIBN (1.2 mg, 7.3 μmol, 0.2 mol eq.). To this a 3 mL MEK solution, in which Sty (1.2324 g, 11.83 mmol, 320 mol eq.) and MAh (1.1588 g, 11.82 mmol, 320 mol eq.) were dissolved, was added to the flask. The contents were degassed with three freeze-evacuate-thaw cycles and polymerised under argon at 60° C. for a prescribed time varying between 5 and 24 hours. Once exposed to air, the polymer was purified by immediate precipitation in diethyl ether, redissolving in DCM and final precipitation in diethyl ether, followed by drying in vacuo overnight (1.6808 g, 70% yield, Ð=1.36).

### Photo-Induced Procedure:

A flask containing a magnetic stirrer bar was charged with BPT (0.0136 g, 0.05 mmol) and a 4 mL dioxane solution containing Sty (1.0481 g, 10.06 mmol, 200 mol eq.) and MAh (0.9867 g, 10.06 mmol, 200 mol eq.). The flask was sealed with a rubber septum and degassed through a three-way stopcock under argon gas flow. The flask was placed about 5 cm away from an LED light source (λₘₐₓ=431 nm) in an aluminium foil-lined box, and the polymerisation carried out at room temperature for a prescribed time. The polymerization was stopped by switching off the light and exposing the mixture to air. The resulting polymer was purified by immediate precipitation in diethyl ether, redissolving in DCM and final precipitation in diethyl ether, followed by drying in vacuo overnight (1.639 g, 80% yield, Ð=1.13). The characterisation for this particular polymer are as follows:

IR (ATR cm⁻¹): 2934 (CH stretching), 1849 (C═O), 1771 (conj. C═O anhydride), 1707 (conj. C═O aldehyde), 1494 & 1454 (CH bending), 701 (CH stretching, benzene derivative). ¹H NMR (400 MHz, DMSO-d₆, δ in ppm): 0.91 (3H, CH₃CH₂CH₂CH₂—), 1.34 (2H, CH₃CH₂CH₂CH₂—), 1.59 (3H, CH₃(CH—)—), 1.80-2.70 (—CHCHAr—), 2.82-3.22 (—CHCHAr—), 3.22-3.95 (—C₂H₂(CO)₂O)—), 5.60-7.60 (ArH). ¹³C NMR (400 MHz, DMSO-d₆, d in ppm): 18.6 (CH₃CH₂CH₂CH₂—), 26.3 (CH₃CH), 35.9 (—CHCHAr—), 41.5 (—CHCHAr—), 50.1 (—C₂H₂(CO)₂O)—), 125.0-175.0 (Ar), 138.0 (C—Ar), 172.5 (—C₂H₂(CO)₂O)—).

### 2. Protocol for Cysteamine Grafting of SMA (SMA-SH)

Stock solutions of polymers were made up in DMF (100 mg/mL), stirred and purged thoroughly with argon. From a freshly prepared cysteamine-HCl stock solution (0.25 M), 0.15, 0.20, and 0.25 molar equivalents ([Cys]₀/[MA]₀) were added and stirred for half an hour. TEA solution (10 mM) aliquots were added dropwise to the reaction mixture via a septum (1:1.1 mol cysteamine-HCl:mol TEA). The reaction was stirred overnight and precipitated in excess driethyl ether. The polymer products were harvested by centrifugation, followed by several cycles of washing with diethyl ether and centrifugation, to remove traces of DMF, and, finally, dried overnight in vacuo.

IR (ATR cm⁻¹): 3400 (NH stretching), 2900 (OH stretching), 2560 (SH stretching), 1849 (cyclic anhydride C═O stretching), 1780 (C═O stretching), 1670 (imide C═O stretching), 1494 & 1454 (CH bending), 700 (CH stretching, benzene derivative). ¹H NMR (400 MHz, DMSO-d₆, δ in ppm): 0.9-2.40 (—CHCHAr—, —NHCH₂CH₂SH), 2.82-3.15 (—CHCHAr—), 3.22-3.95 (—C₂H₂(CO)₂O)—), 5.60-7.60 (ArH), 11.45-12.65 (—NHC). ¹³C NMR (400 MHz, DMSO-d₆, δ in ppm): 32.0-37.5 (—CHCHAr—), 49.5-57.0 (—C₂H₂(CO)₂O)—), 125.0-130.0 (Ar), 141.6 (C—Ar), 172.5 (—C₂H₂(CO)₂O)—), 176.1 (—C₂H₂(CN)₂O)—).

### 3. Ring-Opening and Solubilisation of SMA-SH (SMA-SH_{(aq)})

Suspensions of the SMA-SH polymers were prepared in a 0.01 M borate buffer (pH 8.2), with 10 mM DTT, and refluxed for up to three hours or until a visually homogeneous solution was obtained. The solutions were transferred to dialysis tubing (molecular weight cut-off (MWCO) of 3.5 kDa) and extensively dialysed against 5 mM borate buffer solution with 5 mM DTT. The solutions were lyophilised and stored under argon.

IR (ATR cm⁻¹): 2600-3700 (NH- & OH-stretching), 1640 (imide C═O stretching), 1560 (carboxylic acid C═O stretching), 1400 (carboxylic acid OH bending), 700 (CH stretching, benzene derivative). ¹H NMR (400 MHz, DMSO-d₆, δ in ppm): 0.9-2.40 (—CHCHAr—, —NHCH₂CH₂SH), 2.82-3.15 (—CHCHAr—), 3.22-3.95 (—C₂H₂(CO)₂O)—), 5.60-7.60 (ArH), 11.45-12.65 (—NHC). ¹³C NMR (400 MHz, DMSO-d6, δ in ppm): 32.0-37.5 (—CHCHAr—), 49.5-57.0 (—C₂H₂(CO)₂O)—), 125.0-130.0 (Ar), 141.6 (C—Ar), 172.5 (—C₂H₂(CO)₂O)—), 176.1 (—C₂H₂(CN)₂O)—).

### 4. Procedure to Produce PEG-Bis-SV

Poly(ethylene glycol) ditosylate (PEG-bisTs)

Water was removed from PEGs (1,500 & 3,350 g/mol; 35.0 g) by bulk azeotropic distillation in toluene (300 mL) using a Dean-Stark trap. The two components were mixed and heated to 140° C. Heating was continued until 200 mL of distillate had been collected. The reaction flask cooled to room temperature and any subsequent distillate collected. The remaining mixture was precipitated by dropwise addition into diethyl ether. The free flowing white powder was dried under HVAC overnight. PEG (1,500 g/mol; 15.00 g, 10 mmol, 1 eq & 3,350 g/mol; 33.50 g, 10 mmol, 1 eq) was dissolved in 150 mL DCM in a three-necked round bottom ask. The solution was cooled to 0° C. and TEA (7.0 mL, 50.2 mmol, 5 eq) added dropwise and stirred for an hour. To the rapidly stirred reaction mixture, recrystallized p-toluenesulfonyl chloride (9.533 g, 50.0 mmol, 5 eq) was added in one portion. The mixture was allowed to warm to room temperature and stirred overnight. The reaction mixture was then filtered, and the filtrate concentrated in vacuo prior to precipitation of the polymer with cold diethyl ether. The precipitated solution was centrifuged, redissolved in DCM and precipitated into dry diethyl ether. The precipitate was dried under HVAC overnight affording the poly(ethylene glycol) ditosylate compound. (PEG-bisTs) is a free flowing white powder (PEG-bisTs_{(1,500)}; 16.85 g, 97% & PEG-bisTs_{(3,350)}; 35.16 g, 98%).

IR (ATR cm⁻¹): 2880 (CH stretching), 1460 (CH bending), 1340 (CH bending), 1090 (CO stretching). ¹H NMR (400 MHz, DMSO-d₆, δ in ppm): 2.41 (s, 6H, Ar—CH₃), 3.49 (s, PEG backbone), 4.10 (t, 4H, CH₂SO₂—), 7.48 (d, 4H, J=8 Hz, ArH), 7.78 (d, 4H, J=8 Hz, ArH). ¹³C NMR (400 MHz, CDCl₃, δ in ppm): 21.6 (Ar—CH₃), 68.62 (CH₂CH₂SO₂), 69.3 (CH₂CH₂SO₂), 70.2 (PEG backbone), 128.63 ((p-Ph, CCH₃), 129.9 (m-Ph, CH), 133.0 (o-Ph, CH), 144.8 (CH₂SO₂C—).

Poly(ethylene glycol) diazide (PEG-bisN₃)

PEG-bisTs (PEG-bisTs_{(1,500)}; 15.64 g, 9.00 mmol & PEG-bisTs_{(3,350)}; 32.29 g, 9.00 mmol) and NaN₃ (2.925 g, 45.0 mmol, 5 eq), carefully weighed out, were dissolved in 100 mL dry DMF. The stirred mixture was fitted with a condenser and heated to 90° C. overnight under argon atmosphere. The mixture was cooled down to room temperature and filtrated to remove any residual NaN₃. The mixture was concentrated in vacuo (shielded) and the residue taken up in ethanol and filtered free of any precipitated NaN₃. The solvent was removed, and the residue dissolved in DCM and precipitated into dry diethyl ether and centrifuged.

PEG-bisN_{3(10.500)} was dissolved in 100 mL DCM and washed twice with brine and twice with water. The organic layer was dried over sodium sulphate, reduced to a small volume by rotary evaporation, and finally precipitated into dry diethyl ether. The residue was dried under HVAC overnight to afford a sticky, off-white powder (PEG-bisN_{3(1,500)}; 11.44 g, 82%).

PEG-bisN_{3(3,350)} was purified by dialysis, using 2K MWCO dialysis tubing, and lyophilised resulting in a sticky, off-white powder solid (PEG-bisN_{3(3,350)}; 26.62 g, 87%).

IR (ATR cm⁻¹): 2880 (CH stretching), 2100 (N₃ antisymmetric bending), 1460 (CH bending), 1340 (CH bending), 1280 (CN stretching), 1090 (CO stretching). ¹H NMR (400 MHz, DMSO-d₆, δ in ppm): 3.39 (t, 4H, J=5 Hz, CH₂CH₂N₃), 3.51 (s, PEG backbone), 3.60 (t, 4H, J=5 Hz, CH₂N₃). ¹³C NMR (400 MHz, CDCl₃, δ in ppm): 50.7 (CH₂N₃), 70.1 (CH₂CH₂N₃), 70.6 (PEG backbone).

Poly(ethylene glycol) diamine (PEG-bisNH₂)

PEG-bisN₃ (PEG-bisN_{3(1,500)}; 10.85 g, 7.00 mmol & PEG-bisN_{3(3,350)}; 23.80 g, 7.00 mmol) and PPh₃ (9.18 g, 35.00 mmol, 5 eq) were dissolved in 150 mL MeOH, and the stirred mixture fitted with a condenser and refluxed for 72 hours, under argon atmosphere. The reaction mixture was cooled to room temperature and solvent removed by rotary evaporation. The resulting crude oil was redissolved in DCM and precipitated into dry, cold diethyl ether. After centrifugation, the polymer plug was redissolved in DCM, precipitated, and collected by another cycle of centrifugation. After drying under HVAC overnight the resulting white polymer product, PEG-bisNH₂ was stored under argon (PEGbisNH_{2(1,500)}; 8.808 g, 84% & PEG-bisNH_{2(3,350)}; 20.39 g, 87%).

IR (ATR cm⁻¹): 2882 (CH stretching), 1460 (CH bending), 1340 (CH bending), 1280 (CN stretching), 1090 (CO stretching), 764 (NH wagging). ¹H NMR (400 MHz, DMSO-d₆, δ in ppm): 1.63-1.95 (CH₂NH₂), 2.63 (t, 4H, J=5.2 Hz, CH₂CH₂NH₂), 3.34 (t, 4H, J=5.2 Hz, CH₂CH₂NH₃), 3.51 (s, PEG backbone). ¹³C NMR (400 MHz, CDCl₃, δ in ppm): 41.7 (—CH₂NH₂), 70.6 (PEG backbone), 72.3 (—CH₂CH₂NH₂), 70.6 (PEG backbone).

Poly(ethylene glycol) bis(azanyl-4-oxobutanoic acid) (PEG-bisCOOH)

Dried PEG-bisNH₂ (PEG-bisNH_{2(1,500)}; 8.239 g, 5.50 mmol & PEG-bisNH_{2(3,350)}; 18.41 g, 5.50 mmol) was dissolved in 100 mL THF. To this solution succinic anhydride (2.752 g, 27.5 mmol, 5 eq), dissolved in 20 mL THF, was added dropwise and the solution stirred at 80° C. overnight. The reaction mixture was cooled to room temperature and excess solvent removed by rotary evaporation. The remaining residue was taken up in 10 mL DCM and precipitated with dry diethyl ether. The polymer, PEGbisCOOH, was purified by several reprecipitation cycles with DCM and dry diethyl ether. The final product was a cream-coloured powder (PEG-bisCOOH_{(1,500)}; 6.912 g, 74% & PEG-bisCOOH_{(3,350)}; 15.42 g, 79%).

IR (ATR cm⁻¹): 2882 (CH stretching), 1730 (C═O stretching, carboxylic acid), 1640 (C═O stretching, imide), 1460 (CH bending), 1340 (CH bending), 1280 (CN stretching), 1100 (CO stretching), 764 (NH wagging). ¹H NMR (400 MHz, DMSO-d₆, δ in ppm): 2.30 (m, 4H, —NCOCH₂—), 2.40 (m, 4H, —NCOCH₂H₂—), 3.19 (m, 4H, —CH₂CH₂NH—), 3.39 (m, 4H, CH₂CH₂NH—), 3.51 (PEG backbone), 7.88 (t, —NHCO—), 11.9-12.3 (COOH). ¹³C NMR (400 MHz, CDCl₃, δ in ppm): 28.27 (—NCOCH₂—), 29.87 (—NCOCH₂CH₂—), 39.23 (—CH₂NCO—), 70.32 (PEG backbone), 174.49 (—NCO—), 172.52 (—COOH).

Poly(ethylene glycol bis-succinimidyl valerate) (PEG-bis-SV)

To synthesise the final PEG derivative, PEG-bis-SV, PEG-bisCOOH (PEG-bisCOOH_{(1,500)}; 6.793 g, 4.00 mmol & PEG-bisCOOH_{(3,350)}; 14.19 g, 4.00) was dissolved in anhydrous THF followed by the addition of 5 eq. mol N-hydroxysuccinimide (2.302 g, 20 mmol). Dicyclohexylcarbodiimide (DCC) (4.127 g, 20 mmol, 5 eq), dissolved in a small amount of dry THF, was added dropwise. The solution was left at room temperature, under constant stirring, for two days. The resulting dicyclohexylisourea was removed by filtration, solvent reduced by rotary evaporation, and the polymer precipitated into cold, dry diethyl ether. After centrifugation, the flaky product was redissolved in DCM and precipitated into cold diethyl ether. After drying under HVAC overnight the resulting white polymer product, PEG-bis-SV (PEG-bis-SV_{(1,500)}; 6.888 g, 91% & PEG-bis-SV_{(3,350)}; 14.07 g, 94%) was stored under argon at 4° C.

IR (ATR cm⁻¹): 2882 (CH stretching), 1810 (C═O stretching, anhydride) 1780 (C═O stretching, anhydride), 1730 (C═O stretching, ester), 1640 (C═O stretching, imide), 1460 (CH bending), 1340 (CH bending), 1280 (CN stretching), 1100 (CO stretching). ¹H NMR (400 MHz, DMSO-d₆, δ in ppm): 2.46 (t, —NCOCH₂—), 2.86 (t, —NCOCH₂H₂—), 2.90 (t, N(COCH₂)₂), 3.21 (m, 4H, CH₂CH₂NH—). 3.40 (m, 4H, CH₂CH₂NH—), 3.51 (PEG backbone). ¹³C NMR (400 MHz, CDCl₃, δ in ppm): 28.3 (—NCOCH₂—), 28.8 (N(COCH₂)₂), 29.9 (—NCOCH₂CH₂—), 39.2 (CH₂NCO—), 70.32 (PEG backbone), 174.5 (—NCO—), 174.32 (N(COCH₂)₂)), 172.52 (—COON—).

### Evaluation of SMA-SH Based Hydrogels

Using the above exemplary synthetic procedures, a hydrogel library was prepared using fourteen different SMA-SH polymers with number average molecular weights ranging between 10,650 g/mol and 80,020 g/mol as determined by SEC, and thiol grafting ratios ranging between 0.18 and 0.70 as calculated from FTIR as set out in Table 1; and two PEG-bis-SV crosslinkers prepared from PEG with a number average molecular weight of either 1,500 or 3,350 g/mol, respectively.

TABLE 1
Polymer length scales, degree of functionality and average, estimated distance between
sulfhydryls on SMA polymers.

| | Mₙ | | | | | |
|---|---|---|---|---|---|---|
| Polymer | (g/mol)* | DP** | L_{c}^{¥} | N_{CysH}^{§} | f_{Cys}^{¶} | d̅(nm)^{¤} |
| SMA110 | 10,650 | 53 | 26.1 | 0.18 | 9.5 | 2.7 |
| SMA120 | 10,650 | 53 | 26.1 | 0.22 | 11.7 | 2.2 |
| SMA140 | 10,650 | 53 | 26.1 | 0.40 | 21.2 | 1.2 |
| SMA160 | 10,650 | 53 | 26.1 | 0.58 | 30.7 | 0.8 |
| SMA220 | 36,240 | 179 | 88.2 | 0.20 | 35.8 | 2.5 |
| SMA240 | 36,240 | 179 | 88.2 | 0.56 | 100.2 | 0.9 |
| SMA260 | 36,240 | 179 | 88.2 | 0.68 | 121.7 | 0.7 |
| SMA320 | 52,280 | 396 | 195.1 | 0.23 | 91.1 | 2.1 |
| SMA340 | 52,280 | 396 | 195.1 | 0.41 | 162.4 | 1.2 |
| SMA360 | 52,280 | 396 | 195.1 | 0.70 | 277.2 | 0.7 |
| SMA420 | 80,020 | 396 | 195.1 | 0.25 | 99.0 | 2.0 |
| SMA440 | 80,020 | 396 | 195.1 | 0.48 | 190.1 | 1.0 |
| SMA460 | 80,020 | 396 | 195.1 | 0.61 | 241.6 | 0.8 |
| SMA560 | 263,100 | _(a) | _ (a) | 0.36 | _(a) | _(a) |

*Determined by size exclusion chromatography (SEC) with DMF as eluent and calibrated using polymethylmethacrylate (PMMA) standards.
**Number of calculated repeat units in polymer segment.
^{¥}Modelled using Schrödinger material science's “Polymer builder” function.
${}^{§} Grafting ratio calculated from {N}_{CysH} = \frac{{H}_{Imide}}{( {H}_{Imide} + {H}_{MAh} )} where {H}_{Imide} and {H}_{MAh} are the peak$ heights of the carbonyl stretching of the imide and MAh in FTIR spectra, respectively.
^{¶}Degree of functionality calculated from f_{Cys} = N_{Cys} × DP.
^{¤}Estimated, average distance between sulfhydryl groups on SMA segments, d̅ = L_{c}/f_{Cys}.
^{(a)}Not calculated due to wide dispersity of polymer and inherent associated inaccuracy.

Size exclusion chromatography (SEC) analysis was used to determine the number average molar mass and dispersity of the synthesized polymers. SEC was measured on a Waters™ instrument equipped with a 2487 dual λ UV detector, Waters™ 1515 isocratric HPLC pump, Waters™ 410 differential refractometer (at 30° C.), Waters™ 717 plus autosampler and a Waters™ in-line degasser. The column set-up included a PL-gel 5 μm guard column and two PL-gel 5 μm mixed-C column. DMF was used as an eluent at a flow rate of 1 mL/min. The system was calibrated using low dispersity poly(methyl methacrylate) (PMMA) standards. Sample concentration was 2 mg/mL and all data are reported as PMMA equivalents. Desalting of polymers was achieved using an ÄKTA Pure™ instrument with a HiTrap™ desalting (5 mL) column. UV-Vis Spectroscopy measurements were carried out on a PerkinElmer™ Lambda 20 photodiode array spectrophotometer at room temperature.

The resulting hydrogel library was evaluated based on gelation-, dissolution-, swelling-, and viscoelastic-behaviour. Irreversible dissolution was achieved by exposing the hydrogels to an exogenous, biocompatible cysteine methyl ester (CME)-solution.

### 1. Gelation and Dissolution Studies

Gelation studies were carried out using the two-polymer system: 1) SMA-SH_{(aq)} and 2) PEG-bis-SV. A 30 wt % solution of SMA-SH_{(aq)}, previously dialysed in 5 mM borate buffer (pH=8.2) with 5 mM DTT and lyophilised, is dissolved in UltraPure distilled water. Since the sample is composed of buffer, DTT and SMA-SH_{(aq)}, the weight fraction of polymer in the sample was determined by desalting a sub-sample. Desalting of SMA-SH_{(aq)} was achieved using an ÄKTA pure instrument with a HiTrap desalting (5 mL) column. PEG-bis-SV was made up to a 30% (w/w) polymer solution in phosphate-buffered saline (PBS) at pH 7.4. The molar ratio of thiol to N-succinimidyl succinate was kept at 1:1. The two solutions were injected simultaneously and mixed together in a polytop containing a stirrer bar. Gelation time was started upon injection of the gels into the vial, to imitate the injection conditions in the vas as far as possible, and stopped when the stirrer bar was unable to move freely in the vial. A vial inversion test was done to ensure the formed gels were stable, were able to hold up the stirrer bar, and did not collapse into pieces. All hydrogels formed within minutes (Table 2). The H1 and H2 hydrogel formulations failed the vial inversion test, with the stirrer bar slowly collapsing out of the viscous gel-like structure.

Dissolution times were recorded by injecting a CME-HCl solution (0.25 M, pH adjusted to 8.5) directly into the formed gel and waiting until the stirrer bar moved freely within the dissolving gel. All gelation- and dissolution-times were measured in triplicates (Table 2).

TABLE 2
Gelation and dissolution times of twenty eight hydrogel formulations.

| | | | | PEG-bis-SV crosslinker | | | |
|---|---|---|---|---|---|---|---|
| | | | | PEG-bis-SV_{1,500} | | PEG-bis-SV_{3,350} | |
| | SMA-SH polymer | | | | Dissolution | | Dissolution |
| | Polymer | N_{CysH}^{§} | f_{Cys}^{¶} | Gelation (s) | (min)^{a)} | Gelation (s) | (min)^{a)} |
| H1 | SMA110 | 0.18 | 9.5 | 310 | 9 | 331 | 8 |
| H2 | SMA120 | 0.22 | 11.7 | 306 | 12 | 286 | 10 |
| H3 | SMA140 | 0.40 | 21.2 | 162 | 14 | 151 | 15 |
| H4 | SMA160 | 0.58 | 30.7 | 156 | 23 | 149 | 17 |
| H5 | SMA220 | 0.20 | 35.8 | 164 | 9 | 144 | 8 |
| H6 | SMA240 | 0.56 | 100.2 | 46 | 22 | 47 | 15 |
| H7 | SMA260 | 0.68 | 121.7 | 43 | 22 | 42 | 21 |
| H8 | SMA320 | 0.23 | 91.1 | 49 | 8 | 49 | 11 |
| H9 | SMA340 | 0.41 | 162.4 | 30 | 15 | 30 | 14 |
| H10 | SMA360 | 0.70 | 277.2 | 15 | 25 | 13 | 24 |
| H11 | SMA420 | 0.25 | 99.0 | 32 | 9 | 24 | 9 |
| H12 | SMA440 | 0.48 | 190.1 | 19 | 8 | 16 | 16 |
| H13 | SMA460 | 0.61 | 241.6 | 5 | 14 | 8 | 19 |
| H14 | SMA560 | 0.36 | _(b) | 89 | 14 | 86 | 12 |

${}^{§} Grafting ratio calculated from {N}_{CysH} = \frac{{H}_{Imide}}{( {H}_{Imide} + {H}_{MAh} )} where {H}_{Imide} and {H}_{MAh} are the peak$ heights of the carbonyl stretching of the imide and MAh in FTIR spectra, respectively.
^{¶}Degree of functionality calculated from f_{Cys} = N_{Cys} × DP
^{a)} Dissolution time taken when stirrer bar started to move after injection of CME solution.
^{(b)}Not calculated due to wide polymer molecular weight dispersion.

Twenty eight hydrogel formulations were made up by reacting SMA-SH(aq), with a variety of molecular weights and grafting ratios, with two telechelic crosslinkers, PEG-bis-SV_{1,500} and PEG-bis-SV_{3,350}. The resulting gelation times decreased with increases in the degree of functionality of the SMA-SH polymer as shown in FIG. 2. Clearly there is a relationship between thioester crosslinking and the degree of functionality (f_{Cys}). Dissolution times, which ranged from eight to twenty-five minutes, are related to the permeability of the formed hydrogel and the ability of the CME solution to penetrate the hydrogel's three-dimensional matrix. However, within a hydrogel set, there was a marked increase in dissolution time with increasing degree of cysteamine functionalisation, i.e., decrease in pore size.

### 2. Swelling Behaviour

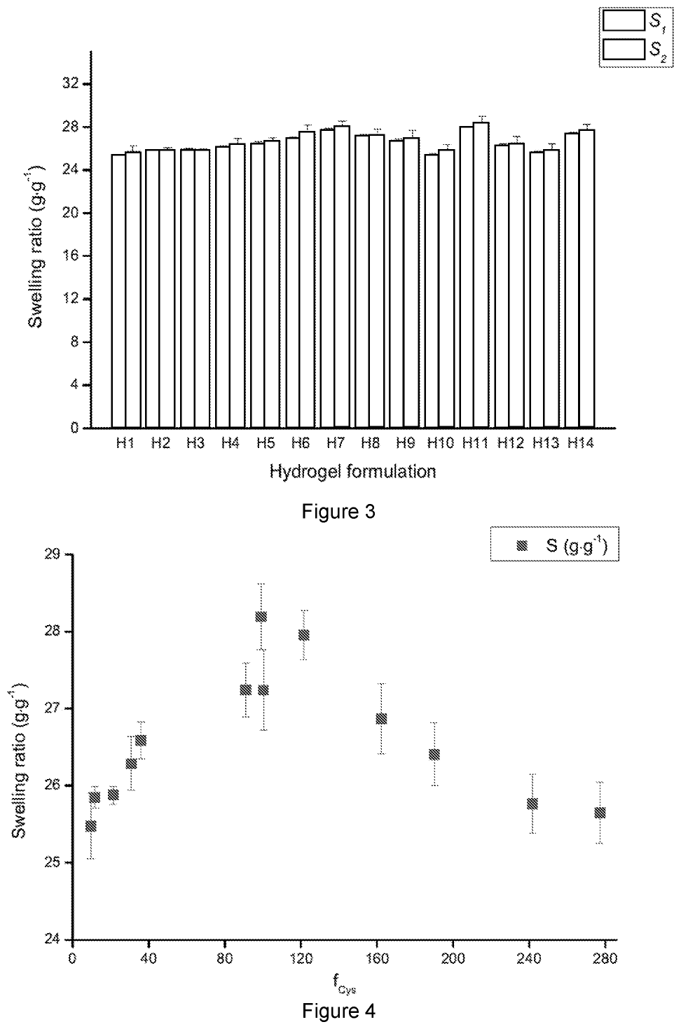

The degree of swelling depends on the crosslinking density, which is, in turn related to the pore size of the hydrogel. Seminal vesicle fluid is mildly alkaline (pH=7.2 to 7.8). The swelling behaviour of the selected hydrogels are investigated using PBS solution (pH=7.4) made up in ultrapure water. It should be noted that swelling behaviour of SMA-based hydrogels is intrinsically linked to pH due to the dissociation of the two protons on the maleic acid residues (pKa₁=3.2 and pKa₂=8.1 at 25° C.). Therefore, further swelling could potentially be achieved in higher pH ranges (pH>8.1). Dried hydrogel films (approximately 5 mm×5 mm×1 mm) were prepared in triplicates from each hydrogel formulation. These films were weighed (W_{d}) and subsequently submerged into the buffer solution for 24 hours. The films were then removed from the buffer solution, excess water was removed from the films by patting the films with filter paper and weighed (Wₛ). The swelling ratios (S) were calculated gravimetrically after wiping excess moisture of the surface of the swollen gels:

$$S = \frac{{W}_{s} - {W}_{d}}{{W}_{d}}$$

where Wₛ is the weight of the swollen hydrogel and W_{d} is the weight of the dry hydrogel. The hydrogels were then dried and re-swollen. The swelling-deswelling cycle weights were averaged and are shown in FIG. 3.

Of the two components of the hydrogel, SMA is the component with the lower water solubility whereas PEG is the one with the higher water solubility. With increasing degree of functionality (f_{Cys}) there is an increase in PEG crosslinker content since the molar ratio of thiol to N-succinimidyl succinate was kept at 1:1. Accordingly, there is a marked increase in swelling ratio, up to a point, until swelling ratios decreases due to higher degrees of crosslinking as shown in FIG. 4.

### 3. Viscoelastic Properties

The hydrogels' viscoelastic behaviours were investigated using amplitude- and frequency-sweep assays. Within the lumen, a hydrogel should be able to undergo deformation and be elastic, but not flow from the vas occlusion site. Rheological studies were used to determine the formed hydrogels' viscoelastic behaviours and compare it to a RISUG/Vasalgel™ equivalent. All rheological tests were performed at 25° C. using a parallel plate rheometer. Ensuring the assays were performed at room temperature allows for comparison of the determined properties to similar dynamic, covalent hydrogel systems. Seminal vesicle fluid pH ranges between 7.2 and 7.8. The hydrogels were made up to 30 w/w % in PBS saline (pH=7.4).

An amplitude sweep test was first performed on the hydrogels to establish their linear viscoelastic region (LVER). Within the LVER, the storage modulus (G′) and loss modulus (G″) remain constant when the amplitude of the applied deformation is changed, also known as the plateau value. Therefore, LVER is the range in which a test can be carried out without destroying the sample. Consequently, parameters measured within the LVER are considered as material characteristics and is a critical input parameter in subsequent frequency-based assays, i.e. frequency sweeps measured within a materials' LVER.

Amplitude sweeps can be used to determine a sample's yield- and flow-point. The yield point is the value of the shear stress at the limit of the LVE region. The flow point is the value of the shear stress at the crossover point, where G′=G″. If a sample shows G″ i G′ in the LVER, it may have a yield point but not a flow point since it is a liquid.

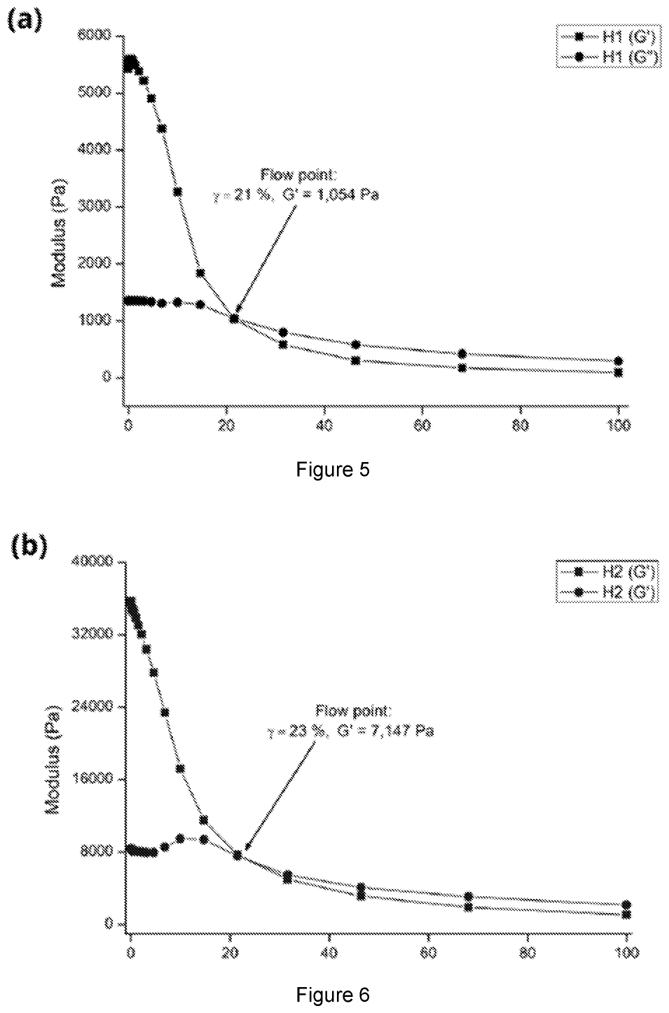

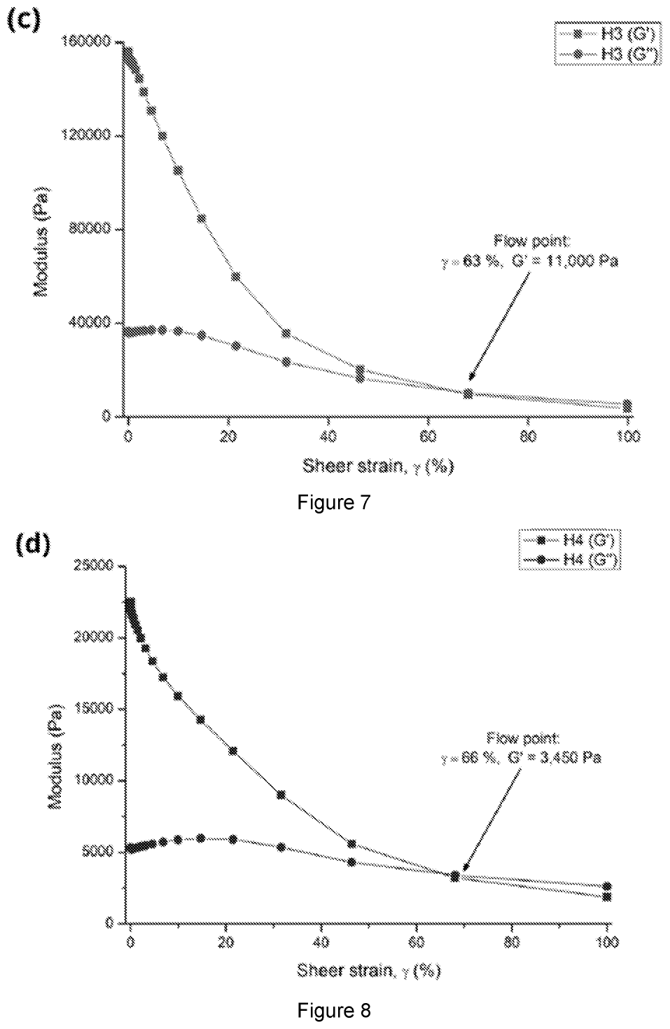

The amplitude sweeps of hydrogels H1 to H4 of Table 2 indicated gel structures, but without strong bonds between the polymers, since they have flow points as is evident from FIGS. 5 to 8. This could be due to the low grafting ratios (N_{CysH}), lower SMA molecular weights (10,650 g/mol) and resulting degree of functionality (f_{Cys}) of SMA110.

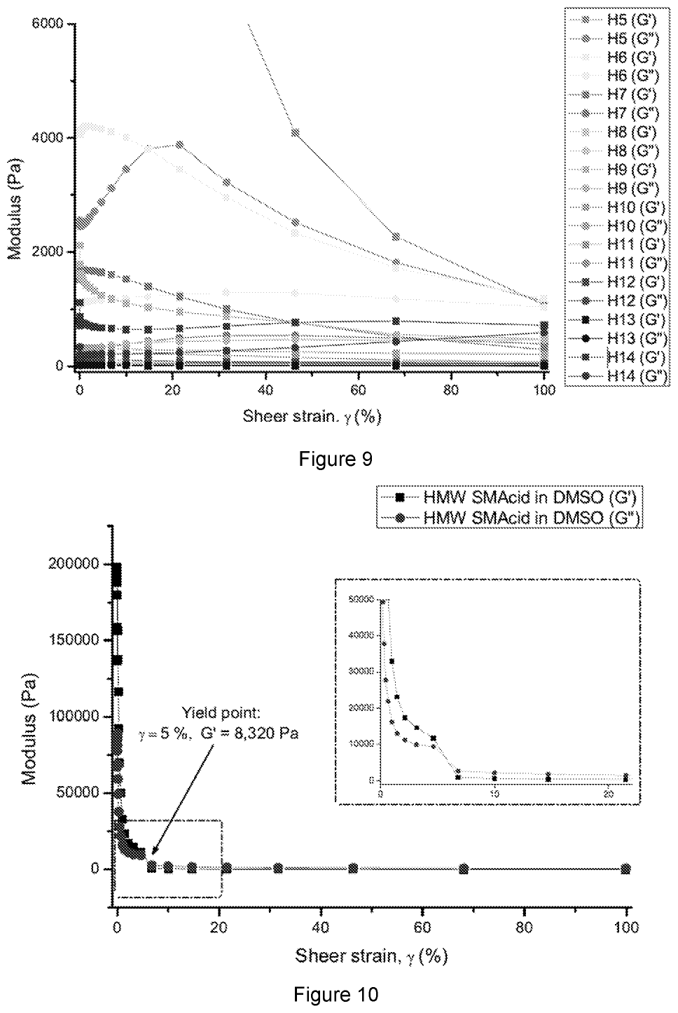

FIG. 9 is the result of the amplitude sweep tests, showing LVER, G′ (elastic modulus=solid nature) and G″ (viscous modulus=liquid nature), as amplitude increases, for H5 to H14. G′ is higher than G″ for all hydrogels, which means the elastic component of the material is dominant over the viscous behaviour, indicating a crosslinked structure. Additionally, there are no flow points observed within the sheer stress amplitude sweep.

The amplitude sweep of the RISUG/Vasalgel™ equivalent (high molecular weight SMAcid) in DMSO is shown in FIG. 10. Note that, for the HMW SMAcid sample, in the LVER, G′<G″. This means that RISUG/Vasalgel™ behaves like a highly viscous liquid and not a gel. This means that there are no strong crosslinks holding the material together.

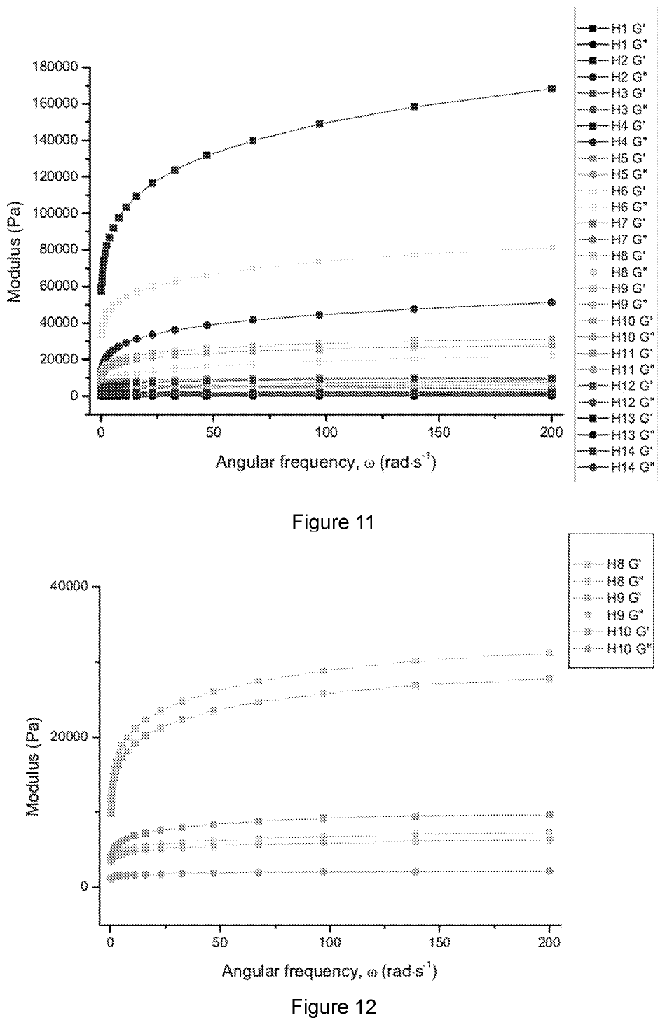

Frequency sweep rheological tests were performed to compare the viscoelasticity of the different formulations (H1 to H14), within their LVERs (sheer strain (δ)=0.1%). FIG. 11 shows storage modulus (G′) and loss modulus (G″) dependencies on angular frequency (ω in rad·s¹). The closer the values of G′ and G″ are to each other, the more crosslinked the samples are. To help visualise trends within a subset of hydrogels, hydrogels formed using SMA 3 (H8 to H10) are shown in FIG. 12. These trends in G′ and G″ show that there is an increase in elastic nature, therefore an increase in crosslinking, with increasing thiol grafting ratio.

The storage- and loss-modulus can be combined to calculate the loss factor (tan δ):

$$tan δ = \frac{{G}^{″}}{{G}^{′}}$$

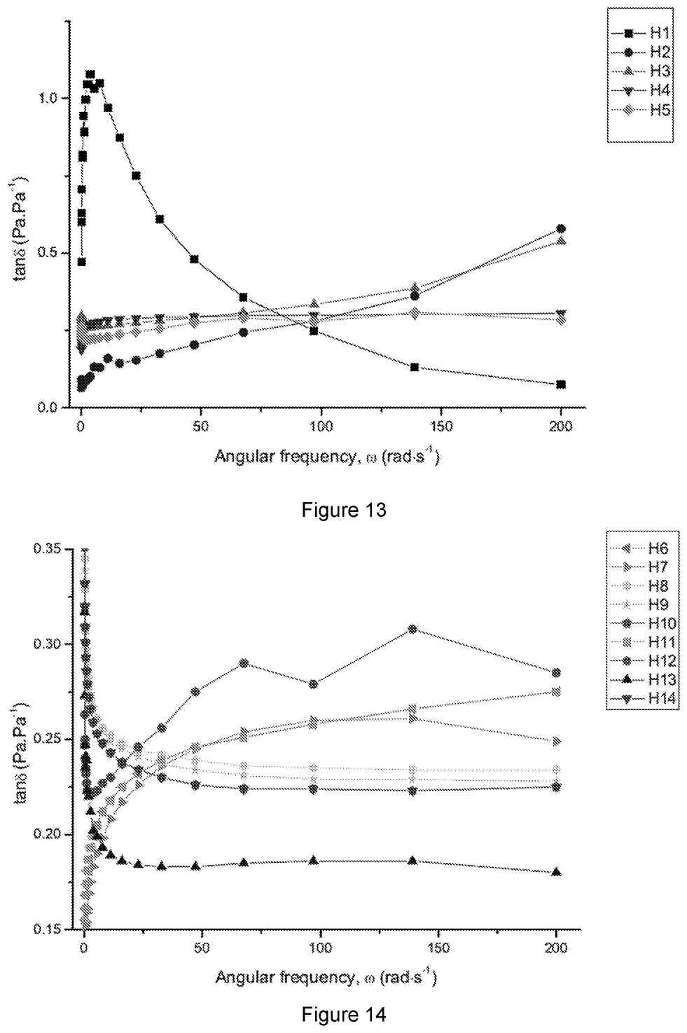

The loss factors (tan δ) were determined with relation to the angular frequency sweeps. The results for H1 to H5 are shown in FIG. 13 and H6 to H14 in FIG. 14. In general, decreasing tan δ values means increasing chemical crosslinking and gel strength. Incorporation of a higher degree of crosslinking into the hydrogels leads to an increase of G′ and its frequency dependence decreases. Moreover, the loss factors for H2 to H14 remain above 0.1 indicating the existence of covalent crosslinks. The smaller the values of tan δ, the more elastic the gels become.

All hydrogel formulations proved to be crosslinked, however, H1 to H5 were weak and had yields points that were identified in amplitude sweep assays. Further frequency-based tests showed that, with increasing crosslinking, the resulting hydrogels behaved more elastically. For all hydrogel formulations, except H1, the storage modulus (G′) was greater than the loss modulus (G″), indicating crosslinked, elastic hydrogels of increasing strength (determined by decreasing tan δ-values) with increasing thiol grafting ratio.

### 4. Hydrogel Permeability

Hydrogel pore size and permeability were investigated by submerging and ultrasonicating the hydrogels in a solution containing silica particles. The silica particles were synthesised using the Stöber method and resulted in monodisperse particles of approximately 2.0 μm. These particles were selected due to their similarity to sperm-head sizes.

Circular hydrogel films (approximately 5 mm×5 mm×1 mm, dry weight between 0.04-0.07 g) were submerged in a 10 w/w % particle suspension in PBS buffer (pH=7.4). Duplicate films were submerged in a PBS buffer solution (pH=7.4), without particles, to serve as reference SEM images. All hydrogel formulations were covered and left at room temperature for 72 hours. Hydrogel suspensions were subsequently ultrasonicated for 5 minutes, to allow for further penetration of the silica particles into the hydrogel matrix. The hydrogel films were thoroughly rinsed with PBS buffer (pH=7.4) and allowed to air dry for an additional 72 hours. All dried films were prepared for SEM, through gold coating, and the resulting micrographs processed with a java-based design program, ImageJ. The diameter of particles found under the surface of the hydrogels were measured.

Five hydrogels were found to be permeable to the silica particles (H1 to H3, H5 and H8) which may indicate an inability to render sperm immotile. The sperm may still be rendered infertile by another mechanism though. It appears that hydrogels with a lower thiol grafting density and subsequently larger pore size, allows for permeation of silica particles. No particles were detected in the other hydrogel formulations (H4, H6 to H7 and H9 to H14).

### 5. Vas Deferens Histology

To determine if the hydrogels had any effect on the morphological- and ultrastructural-features of the male vas deferens, experiments were performed on surgically removed vas. Surgical specimens of vas deferens were obtained from the vasectomies of six adult men. Hydrogels were injected immediately after the specimens were obtained and subsequent reversal of vas occlusion were also carried out on selected samples. After the vas experimental work was completed, the vas deferens samples were prepared for histology assessments. This involved the cryo embedding of the vas in Optimal Cutting Temperature (O.C.T.) compound (a water-soluble blend of glycols and resins that provides a convenient specimen matrix for cryostat sectioning at temperatures of −10° C. and below). The cryo-prepped sample was snap-frozen in liquid nitrogen and sectioned. The samples were mounted on flat-surface specimen blocks and dyed with a haematoxylin and eosin stain (H&E stain). The samples were studied, and images obtained using a Zeiss Axio light microscope. Since two sections of vas were obtained from each patient, one was kept as a reference.

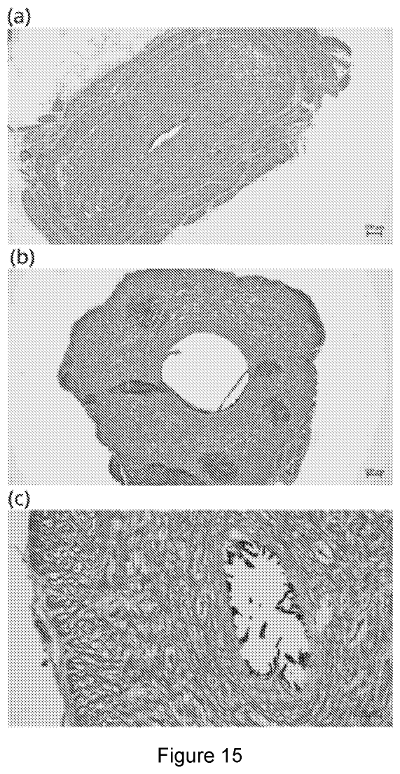

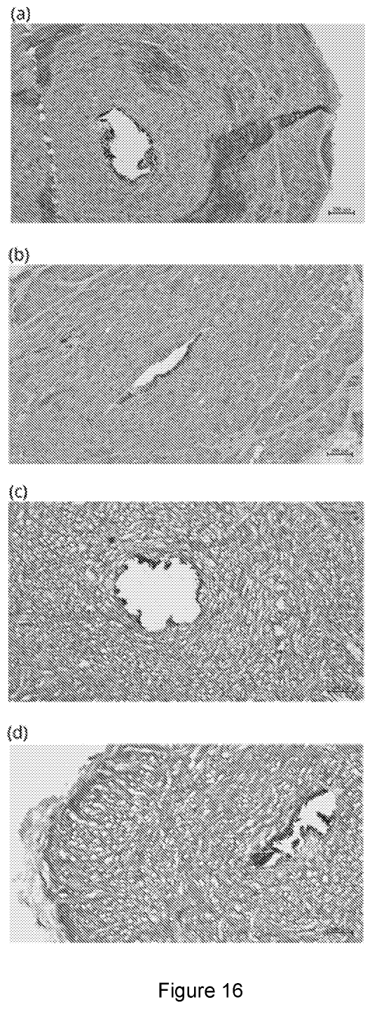

HMW SMAcid, dissolved in DMSO to the specifications of Vasalgel™ (25% solutions by weight) was injected into two samples. The first of these two samples following vas occlusion is shown FIG. 15 (b). After one hour of vas occlusion, the second of these samples was flushed with a sodium bicarbonate solution (2 M, pH=8.0))) to attempt a reversal of the occlusion and is shown in FIG. 15 (c).

Two hydrogel formulations, H9 and H13, were selected for vas studies (FIG. 16: (a) H9 and (c) H13). H9, a slower gelling formulation (30 s) had a faster dissolution time (9 min), in comparison to H13 (5 s and 14 min, respectively). From rheology studies, it was determined that H5 is a more malleable (G′H5>G′H13) in comparison to the rigid H13 formulation (low loss factor (tan δ) across a wide range of frequency sweeps). Both sections of vas were injected with the hydrogel formulations and one vasa sample taken for reversal and subsequent comparison. The differences in rigidity can be seen from the extent that the lumen is stretched by the hydrogel as shown in FIG. 16. After an hour, reversal of the occlusion by hydrogels was brought about by native chemical ligation, irreversibly breaking the thioester crosslinks within the gel. This was achieved by using the vasa with a L-cysteine methyl ester (CME)-solution (0.25 M, pH adjusted to 8.5).

The histograms of FIGS. 15 and 16 show that the RISUG/Vasalgel™ equivalent prepared completely stretches the lumen of the vas, thereby damaging the membrane (dark purple) (FIG. 15, (b)). The harsh reversal conditions destroyed the membrane (FIG. 15, (c)). In comparison, the in situ formation of the SMA-SH based hydrogels, H9 and H13, resulted in the conformation of the hydrogel to the highly folded shape of the lumen. Reversal of both H9 and H13 resulted in the membrane of the lumen remaining intact (FIGS. 16; (b) H9 reversal, and (d) H13 reversal).

Advantageously, the polymer system for forming hydrogels is based on reversible chemistry and low molar mass polymers. Existing contraceptive gels used in vas occlusion such as Vasalgel™ and RISUG are more like polymer plugs and are not necessarily fully reversible or safe. The main differences between the polymer system described herein and Vasalgel™ and RISUG are: a) the use of aqueous solutions as starting materials; b) the ability to crosslink the hydrogel in situ in a covalent and thus stable manner and 3) the readily reversibility of the crosslinking to revert back to low viscous material that can be removed from the body more easily. In use, the polymer system described herein provides a contraceptive that is fully reversible, due to the incorporation of dynamic covalent bonds into the hydrogel system. Chemical crosslinking of the polymer forms a robust hydrogel that can be “uncrosslinked” in a short time span to achieve reversal of the contraceptive effect. The administration of the hydrogel may also be minimally intrusive due to the ability to administer aqueous solutions rather than viscous polymer materials. Reversibility of the hydrogel is benign and quick (i.e., within minutes) and occurs without damaging the morphological- and ultrastructural-features of the vasa. The hydrogels exhibited fast gelation times and conformed to the highly folded nature of the lumen.

The foregoing description has been presented for the purpose of illustration; it is not intended to be exhaustive or to limit the invention to the precise forms disclosed. Persons skilled in the relevant art can appreciate that many modifications and variations are possible in light of the above disclosure. For example, it would be apparent to those skilled in the art that another type of dynamic covalent crosslinking reaction can be used to form the hydrogel. The biologically compatible at least partially thiolated SMA-SH polymer can also participate in crosslinking reactions with differently functionalized cross-linking agents, provided they are compatible with aqueous media. The reversibility of the hydrogel formation may, for example, also be based on thiol-disulfide exchange with dynamic S—S bonds in the crosslinked matrix or be based on thia Micheal addition (click reaction between a thiol and an ene compound with an electron-withdrawing group such as an acrylate). In the case of the formation dynamic S—S bonds, the hydrogel should be formed ex vivo prior to injection, and the dissolution agent may be dithiothreitol (DTT). In the case of thia-Michael exchange the hydrogel may be formed in situ at the target site and the dissolution agent may be glutathione, for example.

It will be apparent to those skilled in the art that the polymer system described herein, which is configured to form a dynamic and biocompatible hydrogel, may find use in any appropriate biological or medical application where “on-demand” reversibility of hydrogel formation is required or advantageous. Its applications are not necessarily limited to reversible male contraception. For example, the hydrogel may also find use as a reversible female contraceptive and thus be administered within the vagina as a vaginal gel or on the cervix as a cervical cap to prevent or obstruct the movement of sperm to avoid fertilization.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. A polymer system for forming a hydrogel comprising
a water-soluble and at least partially thiolated poly(styrene-co-maleic acid) polymer; and
a water-soluble cross-linking agent with reactive functional groups at its ends which are configured to crosslink to the thiol groups of the thiolated poly(styrene-co-maleic acid) polymer when aqueous solutions of the at least partially thiolated poly(styrene-co-maleic acid) polymer and the cross-linking agent are combined to form the hydrogel in use.

2. The polymer system as claimed in claim 1, wherein the reactive functional groups are activated esters.

3. The polymer system as claimed in claim 2, wherein the activated esters are succinimidyl valerate groups or pentafluorophenyl ester groups.

4. The polymer system as claimed in claim 1, wherein the cross-linking agent is a biocompatible, water-soluble telechelic polymer.

5. The polymer system as claimed in claim 4, wherein the telechelic polymer is poly(ethylene glycol bis-succinimidyl valerate).

6. The polymer system as claimed in claim 5, wherein the poly(ethylene glycol bis-succinimidyl valerate) has a number average molecular weight of between 1,500 and 3,500 g/mol.

7. The polymer system as claimed in claim 1, wherein the at least partially thiolated poly(styrene-co-maleic acid) polymer has a number average molecular weight of between 10,000 and 85,000 g/mol.

8. The polymer system as claimed in claim 1, wherein the at least partially thiolated poly(styrene-co-maleic acid) polymer has a narrow molecular weight distribution characterised by a dispersity (Ð) of less than 2.

9. The polymer system as claimed in claim 1, wherein the at least partially thiolated poly(styrene-co-maleic acid) polymer is at least partially thiolated poly(styrene-alt-maleic acid) polymer and the thiol grafting ratio of cysteamine to maleic acid of the thiolated poly(styrene-alt-maleic acid) polymer is between 0.1 and 0.8.

10. The polymer system as claimed in claim 1, wherein the at least partially thiolated poly(styrene-co-maleic acid) polymer and the cross-linking agent are each independently present in separate aqueous solutions prior to use.

11. (canceled)

12. A hydrogel formed from the polymer system as claimed in claim 1.

13. (canceled)

14. A method of preparing a polymer system as claimed in claim 1, the method comprising the steps of
thiolating a poly(styrene-co-maleic anhydride) polymer with a thiolating agent that possesses both a primary amine group and an SH-group to form an at least partially thiolated poly(styrene-co-maleic anhydride) polymer;
hydrolysing the at least partially thiolated poly(styrene-co-maleic anhydride) polymer to form the at least partially thiolated poly(styrene-co-maleic acid) polymer; and separately providing the cross-linking agent.

15. The method as claimed in claim 14, wherein cysteamine hydrochloride is used to modify the poly(styrene-co-maleic anhydride) polymer, where the molar ratio of cysteamine to maleic anhydride used during modification ranges between 0.1:1 and 0.8:1.

16. The method as claimed in claim 14, wherein the poly(styrene-co-maleic anhydride) polymer is synthesised by controlled radical polymerisation to obtain a narrow molecular weight distribution characterised by a dispersity (Ð) of less than 2.

17. The method as claimed in claim 14, wherein the cross-linking agent is poly(ethylene glycol bis-succinimidyl valerate).

18. A method of vas-occlusive contraception, the method comprising providing a hydrogel formed from the polymer system as claimed in claim 1 in a lumen of a vas deferens in a living subject.

19. The method as claimed in claim 18, further comprising optionally administering a solution into the lumen that is configured to dissolve the hydrogel present in the lumen to reverse the occlusion of the lumen.

20. The method as claimed in claim 18, wherein aqueous solutions of the at least partially thiolated poly(styrene-co-maleic anhydride) polymer and the cross-linking agent are separately or concurrently administered into the lumen to reversibly crosslink into a contraceptive hydrogel in the lumen.

21. (canceled)

22. (canceled)

23. A kit for forming a hydrogel comprising the polymer system as claimed in claim 1.

24. The kit as claimed in claim 23, wherein the at least partially thiolated poly(styrene-co-maleic acid) polymer and the cross-linking agent of the polymer system are each independently present in separate aqueous solutions.

25. (canceled)
